(19) United States
(12) Patent Application Publication
(10) Pub. No.: US 2001/0033825 A1
(43) Pub. Date: Oct. 25, 2001

(54) Method of ultrasonic coupling agent

(76) Inventor: Ted E. Douglas, Hot Springs, AR (US)

Correspondence Address: Ted E. Douglas, 100 Hillcrest St, Hot Springs, AR, 71901, US

(21) Appl. No.: 09/835,928

(22) Filed: Apr. 16, 2001

Related U.S. Application Data
(60) Provisional application No. 60/197,636, filed on Apr. 17, 2000.

(51) Int. Cl. A61K 49/22

(52) U.S. Cl. 424/9.5

(57) ABSTRACT

An improved method for formulating a couplant for transmission of ultrasound therapy or sonic imaging consisting of a super absorbent polymer which rapidly hydrates when added to water thus allowing transmission of ultrasonic therapy or sonogram imaging when resulting hydrated polymer gel is applied to the skin of a mammal, and the appropriate ultrasonic or sonogram imaging device is used.

This application is a continuation-in-part of Provisional Application No. 60/197,636 entitled “Method of Ultrasonic Coupling Agent” filed on Apr. 17, 2000.

An improved method for formulating a couplant for transmission of ultrasound therapy or sonic imaging consisting of a super absorbent polymer which rapidly hydrates when added to water thus allowing transmission of ultrasonic therapy or sonogram imaging when resulting hydrated polymer gel is applied to the skin of a mammal, and the appropriate ultrasonic or sonogram imaging device is used.

### BACKGROUND OF THE INVENTION

The present invention relates to a coupling agent for the application of electrical and ultrasonic energy and/or sonogram imaging. Electrical and ultrasonic energy therapies are old and well known techniques in medical therapy or physical rehabilitation. Sonogram imaging is also a well known and widely used medical practice. These devices involve the application of a transducer element to the skin of the subject to effectively transfer the impulse or energy. These techniques require a coupling agent to assure proper contact is maintained between the transducer and the skin of the subject. Numerous compositions have been used as coupling agents, including mineral oil, water, glycerin, propylene glycol, acrylic polymers, carboxy alkyl cellulose, hydroxyethylcellulose, parabens, organic salts, aloe vera and various combinations of these ingredients. Water and mineral oil are two of the best coupling agents, however both have major limitations. In order to use water, the area of the skin to be imaged or treated must be submerged in water, and a special waterproof transducer must be used. This usually limits the area being treated to a hand or limb. While being an excellent coupling agent, mineral oil is difficult to remove from both the subject and transducer, and also leaves an objectionable “oily feeling” to the subject.

It is important that the coupling agent have good wetting characteristics in order to provide good transfer of ultrasonic or imaging energy. The coupling agent should contain no ingredient that can cause an allergic reaction or irritation to the skin of the subject and should be non-staining. It also should be easily washed or removed from both the subject and transducer, preferably water-soluble.

One of the most important aspects of the coupling agent regards viscosity. Since the application of ultrasonic or imaging energy generates heat, the coupling agent must maintain viscosity in order to allow contact between the skin of the subject and the transducer.

Prior art teaches that ultrasound and sonic imaging coupling agents are formulated and delivered to the end user either fully hydrated or the form of a hydrogel sheet. Prior art also teaches the use of polyacrylamide and polyacrylate as thickeners. Montecalvo U.S. Pat. No. 5,727,550 teaches the use of polyacrylamide as a thickener in the construction of an ultrasonic couplant in sheet form. Larson, et al. U.S. Pat. No. 6,039,694 teaches the use of acrylate derivatives to construct a coupling sheath for ultrasound transducers. The present invention is an improvement over these inventions in that these thickeners require multiple additional ingredients or components, and do not have the capability of the present invention to rapidly hydrate to a usable state at room temperature without the need for agitation or mixing with anything other than tap water. Due to the weight and container size of prior art couplants, they are therefore expensive to ship and store. Some coupling agents incorporate hydrophillic acrylates and/or acrylamides, however these agents require mechanical mixing, agitation and/or heat to complete the hydration process to a usable form. The present invention is a combination of super absorbent acrylamide and sodium acrylate copolymer suspended in mineral oil with the unique ability to rapidly hydrate from concentrate form at room temperature to a useful couplant form within five minutes or less, completely dispersing without agitation or mixing. The present invention involves a cosmetic grade super absorbent polymer, which can be delivered to the end user in a dehydrated state, thus saving considerable expense in shipping and storage. 4 ml of super absorbent polymer concentrate dispersed in medical grade mineral or white oil, will hydrate 355 ml of tap water a ratio of one to 87.5. The hydrated solution meets all the critical requirements for ultrasound and sonic imaging coupling agents. Further, the hydrated coupling agent of the present invention exceeded the performance of all other coupling agents to which it was compared.

### SUMMARY OF THE INVENTION

The present invention provides an improved ultrasound and sonic imaging coupling agent. More specifically, it is the object of the present invention to provide a method of formulating a super absorbent polymer concentrate suspended in mineral or white oil which hydrates rapidly (less than five minutes) when added to water thus allowing a coupling agent for transmission of ultrasonic therapy or sonogram imaging by the end user and providing considerable savings in shipping and storage costs. SNF Floerger of St. Etienne, France manufactures an acrylamide/sodium acrylate super absorbent copolymer in cosmetic grade which is the basis for the present invention. Using 4 ml of the concentrate super absorbent copolymer solution and adding 355 ml of water rapidly creates a thick gel. The hydrated gel has a superior viscosity under heat when compared to all other couplants tested. Bunnell et al. U.S. Pat. No. 3,710,779 teaches a ultrasonic coupling agent. When the present invention was compared to the '779 patent, the present invention retained its' viscosity 67 percent longer, thus allowing for the use of less couplant on the subject. Buchalter U.S. Pat. No. 4,002,221 teaches a coupling agent. When the present invention was compared to the '221 patent, the present invention retained its' viscosity 60 percent longer, again allowing for the use of less couplant. Unger, et al. U.S. Pat. No. 5,948,387 teaches a contrast media for ultrasonic imaging. When the present invention was compared to the '387 patent, the present invention retained its' viscosity 52 percent longer, again allowing for the use of less couplant. The present invention was also compared to various other water soluble couplant formulas bearing no patents, and in each comparison study it out performed all formulas by retaining its' viscosity at least 48 per cent longer than all water soluble formulas to which the present invention was compared. In each test an 9 inch by 9 inch square area was clearly marked on the back of a human male test subject. A Mettler Electronics, Corp. Model 730 Sonicator Ultrasound equipment was used in all tests. The ultrasound equipment was set for 15 minutes of treatment duration, with identical frequency settings of 14 watts, using the same transponder. Each test was started using 20 cc of couplant formula. When the ultrasound equipment indicated loss of delivery of ultrasound to the subject by audible and visual guides, the time was recorded. A total of 11 commercially available couplant formulas were tested.

An advantage of the present invention is that it outperformed all other formulas to which it was compared by maintaining a minimum of 48 percent longer time for transmission of ultrasonic treatment, thus requiring less couplant to be applied to the test subject resulting in considerable cost savings to the medical practitioner. Another advantage of the present invention is its' superiority in maintaining viscosity under heat generated by the ultrasound treatment. It was noted that several of the formulas lost viscosity and the medical practitioner was required to use toweling to wipe the couplant from the side of the test subject. In some tests, the couplant lost viscosity to such an extent that it ran down the sides of the test subject, thus wetting the bedding on the treatment table. In all tests of the 11 couplants, additional gel was required to complete the treatment. The present invention required no additional couplant. Yet another advantage of the present invention is that being 98.9 per cent water in its' hydrated state, it was easily cleaned from both the test subject and the transducer at the conclusion of the test. Another advantage of the present invention is that it can be packaged in a collapsible container thus being additionally cost effective in shipping and storage. Another advantage of the present invention is that in its' hydrated state of 98.9 per cent water it is unlikely to cause any skin irritation or allergic reactions. An additional advantage of the present invention is that being 98.9 per cent water at time of use it is an excellent medium for conduction ultrasound and sonogram imaging, as water has proven to be one of the best conductors of ultrasound and sonic imaging. Another advantage of the present invention is that it is non-staining, and was pleasing to the test subject, as it contains only a minute non-detectable amount of medical grade mineral oil. Another advantage of the present invention is that being 98.9 per cent water it maintained throughout the treatment an acoustic impedance approximately equal to human tissue, thereby affording a near perfect couplant medium for ultrasound and sonic imaging signals. Another advantage of the present invention is that should the couplant become dry during the treatment, it can be rehydrated by the application of water instead of additional couplant gel, thus being even more cost effective to the medical practitioner. Still another advantage of the present invention is the hydrated couplant requires no preservatives or additives, when used in a normal length of time (two to three weeks). Another advantage of the present invention is the couplant does not show significant changes is viscosity at high temperature, and remained viscous at temperatures much higher than allowable for skin contact. Still another advantage of the present invention is the super absorbent copolymer concentrate will hydrate when added to water of any temperature, although best results will be achieved at water temperatures from 75 to 104 degrees F., allowing the couplant to hydrate at room temperature. A further advantage to the present invention is that comprehensive toxicological testing determined it to be non-toxic by ingestion; non-irritating and non-sensitizing to the skin or eyes. A two-year feeding study on rats did not reveal adverse health effects. A two-year feeding study on dogs did not reveal adverse health effects. Testing conducted according to the Draize technique showed the material produces no corneal or iridial effects and only slight transitory conjuctival effects similar to those which most foreign matter have on conjunctivae. The results of testing on rabbits showed this material to be non-irritating to the skin. The results of oral testing on rats LD50/oralrat>5000 mg/kg showed this material to be non-toxic even at high dose levels. The results of testing on human volunteers (Human Patch Test) showed this material to be non-irritating to the skin. The results of in vitro testing on cell cultures showed this material to be only moderately irritating to the eyes.

I claim:

1. An improved method of providing a couplant allowing the transmission of ultrasonic or sonogram images consisting essentially of:
a rapid hydrating super absorbent acrylamide/sodium acrylate copolymer concentrate suspended in medical grade mineral oil with an active content of about 57.5 percent with a viscosity as supplied 400-1,400 cps (Brookfield LVT sp.3:30 rpm) and a viscosity in a 1 percent solution of 10,000-15,000 cps (Brookfield RVT sp 6:50 rpm) and a pH in a 1 percent solution of 5.8-6.2 having the ability to hydrate in tap water with a pH of about 4.8 to 8.2 within five minutes after combining the water and the copolymer concentrate and requiring no additional thickeners, preservatives or ingredients.
